(19) United States
(12) Patent Application Publication
(10) Pub. No.: US 2017/0105702 A1
(43) Pub. Date: Apr. 20, 2017

(54) ULTRASONIC PROBE

(71) Applicant: NIHON DEMPA KOGYO CO., LTD., Tokyo (JP)

(72) Inventor: YOJI NAKA, SAITAMA (JP)

(73) Assignee: NIHON DEMPA KOGYO CO., LTD., Tokyo (JP)

(21) Appl. No.: 15/039,395

(22) PCT Filed: Mar. 11, 2015
(86) PCT No.: PCT/JP2015/057141

§ 371 Date: May 25, 2016

(30) Foreign Application Priority Data
Mar. 27, 2014 (JP) 2014-064959

(51) Int. Cl. A61B 8/00; A61B 8/08; G10K 11/35

(52) U.S. Cl. CPC A61B 8/4461; G10K 11/355; A61B 8/483

(57) ABSTRACT

An ultrasonic probe having: an ultrasonic transmission and reception unit provided inside housing; and a drive device provided therein that encases a main sound transmission medium and swings the ultrasonic transmission and reception unit. The ultrasonic probe is characterized by: the drive device being a drive transmission mechanism that converts the rotation of a drive motor to swinging of the ultrasonic transmission and reception unit; all or part of the drive transmission mechanism comprising a gear mechanism; and preventing backlash in a meshing section of at least a pair of gears in the gear mechanism, by elastically impelling and pressing one pair of gears on to the other pair of gears by using compression springs.

### TECHNICAL FIELD

The present invention relates to a short-axis oscillating ultrasonic probe that transmits and receives ultrasonic waves from a piezoelectric element group which is an ultrasonic transmission and reception unit for a subject (living body) and that takes in three dimensional (3D) data for ultrasonic diagnosis of the subject, and more particularly relates to an ultrasonic probe that prevents backlash occurring during meshing of tooth surfaces of a pair of gears which mechanically oscillate a piezoelectric element group of the ultrasonic probe in a short axis direction.

### BACKGROUND ART

An ultrasonic diagnostic device using a mechanical short-axis oscillating ultrasonic probe for taking in three dimensional data generally creates a three dimensional image based on drive signals of a drive motor which is used for oscillating a piezoelectric element group or output signals of an encoder provided at a motor drive mechanism.

However, in the both cases, the ultrasonic transmission and reception unit (the piezoelectric element group) that is a subject to drive is arranged in a housing (a sealed container) which contains and seals acoustic propagation liquid, e.g., oil. On the other hands, the driving motor and the encoder are arranged outside the housing in order to prevent from directly contacting the acoustic propagation liquid. Therefore, the ultrasonic transmission and reception unit is driven through the driving motor or the encoder by a gear mechanism provided there between, e.g., made of a pair of bevel gears. Such a gear mechanism has a problem that if backlash of gears meshing each other is larger than a prescribed value, the deviation may occur in an ultrasonic image to be created when the ultrasonic transmission and reception unit oscillates.

That is, ultrasonic images of the subject are taken in both cases that the ultrasonic transmission and reception unit (piezoelectric element group) oscillates in one direction (forward direction) and oscillates in a reverse direction (the other direction). Even though ultrasonic images are created based on drive signals of the drive motor or output signals of the encoder as it is determined that the ultrasonic transmission and reception unit is in the same oscillating angle in a forward direction and a reverse direction, the ultrasonic transmission and reception unit is actually in different oscillating positions (angles) in the forward and reverse directions by backlash of gears meshing each other that forms a gear mechanism for oscillation. In result, the above-described deviation occurs in the ultrasonic image.

Therefore, conventionally, as shown in FIGS. 9 (a) (b), in the short-axis oscillating probe, a piezoelectric element group 320 arranged in the longitudinal direction which has an acoustic lens on an ultrasonic transmission and reception surface is provided on a rotary holding table 310 contained in a sealed container 300, and the piezoelectric element group 320 is oscillated in its short-axis direction through a drive shaft 307 and bevel gears 308, 309 so as to mechanically scan ultrasonic waves transmitted and received from the ultrasonic transmission and reception surface of the piezoelectric element group 320. Liquid as acoustic medium L is filled and sealed in the sealed container 320 by covering with a cover 330.

Here, backlash of gears 308 and 309 meshing each other is adjusted appropriately by rotating a pair of holding shafts 314 screwed in upper parts of both ends of the rotary holding table 10, e.g., with a tip of a screw driver to be inserted into an adjusting groove 314a (see Patent Document 1).

For the conventional adjustment of backlash of gears 308, 309 meshing each other as described above, an ultrasonic probe having acceptable limit of backlash is prepared as a limit sample, and an operator rotates and oscillates the limit sample of ultrasonic probe by hand and determines by the feel whether backlash is in the acceptable range.

Further, as other conventional example, as show in FIGS. 10(a), (b), an ultrasonic probe has two-divided gears, a driving bevel gear 401 and a driven bevel gear 402, between an oscillation device and a motor shaft 408 which oscillate the oscillation device and to which the driving bevel gear 401 is fixed. The bevel gears 401 and 402 are supported by the motor shaft 408 so that one of the bevel gears 401 and 402 is rotatable relative to the other and biased to the other in one direction by coil springs 405 attached to pins 403 and 404.

With such structure, tooth surfaces of the driving bevel gear 401, together with tooth surfaces of the driven bevel gear 402 adjacent to the bevel gear 401, push both sides of tooth surfaces of a bevel gear 430 of the other end to mesh therewith by the strength of the coil springs 405, thereby eliminating backlash between tooth surfaces.

### CITATION LIST

### Patent Documents

Patent Document 1: Unexamined Japan Patent Application No. 2012-95256

Patent Document 2: Unexamined Japan Patent Application No. 1990-177043

### SUMMARY

### Technical Problems

However, such backlash elimination of the drive gear mechanism of the conventional ultrasonic probe, it is conceivable to minimize backlash by adjusting the space of the gear tooth meshing each other but there is a limit to keep eccentricity accuracy of the gear below a specified value. Therefore, even if it is possible to eliminate the backlash at “the certain oscillating position” of the ultrasonic transmission and reception unit (piezoelectric element group), backlash may occur at “other oscillating positions”. Because of this reason, it was technically impossible to eliminate backlash over the entire oscillating range of the drive gear mechanism. Further, since it takes many working steps to adjust backlash, it prevents decrease of manufacturing cost of the ultrasonic probe, which is a problem (in the case of the conventional example in the Patent Document 1).

Further, there is a problem in the case of conventional example disclosed in the Patent Document 2 that since the bevel gear forming the gear mechanism to be used in the oscillation of the ultrasonic transmission and reception unit is divided in two, the bevel gear becomes large, which hinders downsizing of the ultrasonic probe.

### Solutions to Problems

In order to solve the above problems, an ultrasonic probe of the present invention, comprises an ultrasonic transmission and reception unit provided inside a housing, an acoustic transmission medium sealed in the housing, and a drive device for oscillating the ultrasonic transmission and reception unit. The drive device is a drive transmission mechanism for converting rotation of a drive motor to oscillation of the ultrasonic transmission and reception unit. All or a part of the drive transmission mechanism comprises a gear mechanism. In a meshing section of at least one pair of gears in the gear mechanism, one of the pair of gears is elastically biased and pressed to the other of the pair of gears.

Further, in an ultrasonic probe of the present invention, the one of the pair of gears, together with other member that rotates integrally therewith is elastically biased and pressed to the other of the pair of gears.

Furthermore, in an ultrasonic probe of the present invention, the pair of gears is bevel gears meshing each other.

Furthermore, in an ultrasonic probe of the present invention, the other member that rotates integrally with the one of the pair of gears is a drive shaft for transmitting rotational force to the one of the pair of gears, or a rotary shaft of the gear mechanism.

In an ultrasonic probe of the present invention, a member for merging and pressing the one of the pair of gears to the other of the pair of gears is a compression spring provided around the other member that rotates integrally with the one of the pair of gears.

### Advantageous Effects of Invention

According to the present invention, backlash between tooth surfaces of a pair of gears meshing each other is prevented with simple structure, and deviation due to the oscillation of the ultrasonic transmission and reception unit does not occur in ultrasonic images to be created, and assemblability of the ultrasonic probe is superior.

### BRIEF DESCRIPTION OF THE DRAWINGS

FIG. 1 shows a front view (a) and a side view (b) of an ultrasonic probe of the present invention.

FIG. 2 shows a cross sectional view of the ultrasonic probe of the present invention, taken along II-II of FIG. 1 (b).

FIG. 3 shows a perspective view of an ultrasonic transmission and reception unit and an oscillating unit of the ultrasonic probe shown in FIG. 1 of the present invention.

FIG. 4 shows a perspective view of a whole oscillating unit for the ultrasonic transmission and reception unit of the ultrasonic probe of the present invention shown in FIG. 3.

FIG. 5 is an enlarged view of the first embodiment of a gear mechanism of the oscillating unit for the ultrasonic transmission and reception unit shown in FIG. 4.

FIG. 6 is an enlarged view of an area pointed by an arrow A in FIG. 5

FIG. 7 is an enlarged view of the second embodiment of a gear mechanism of the oscillating unit for the ultrasonic transmission and reception unit shown in FIG. 4.

FIG. 8 is a cross sectional view of a bearing pointed by an arrow B shown in FIG. 7 which shows the second embodiment of the gear mechanism of the oscillating unit for the ultrasonic transmission and reception unit shown in FIG. 4.

FIG. 9 shows a conventional probe; (a) shows a perspective view of the ultrasonic probe with a cover removed, seen from the above; (b) shows a cross sectional view of the ultrasonic probe in which acoustic propagation liquid is contained and sealed with a cover.

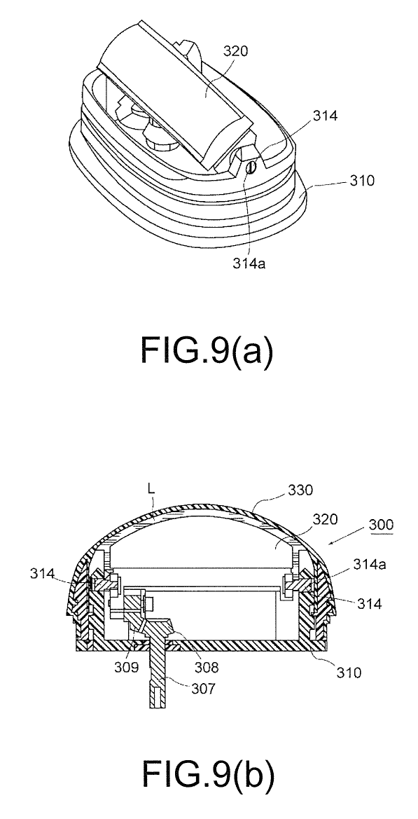

FIG. 10 shows an oscillating mechanism of an oscillating device of another conventional ultrasonic probe; (a) shows a cross sectional view thereof; (b) shows a plan view thereof seen from the above.

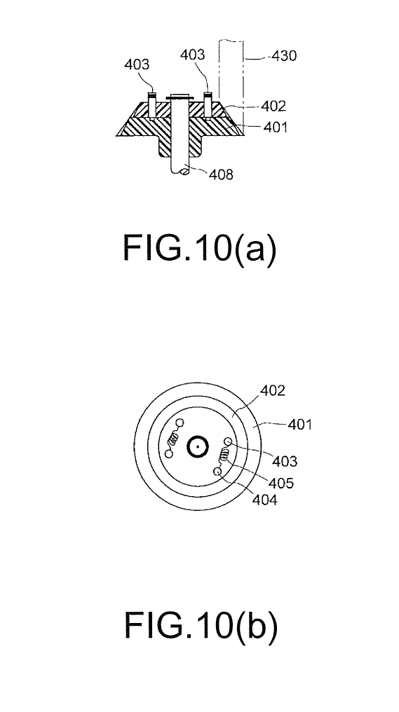

### DESCRIPTION OF EMBODIMENTS

### First Embodiment

The first embodiment of an ultrasonic probe of the present invention will be described with accompanying drawings in the following.

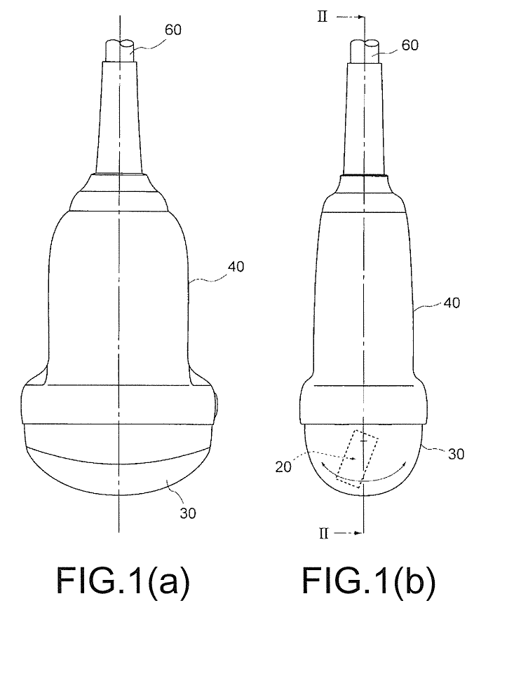

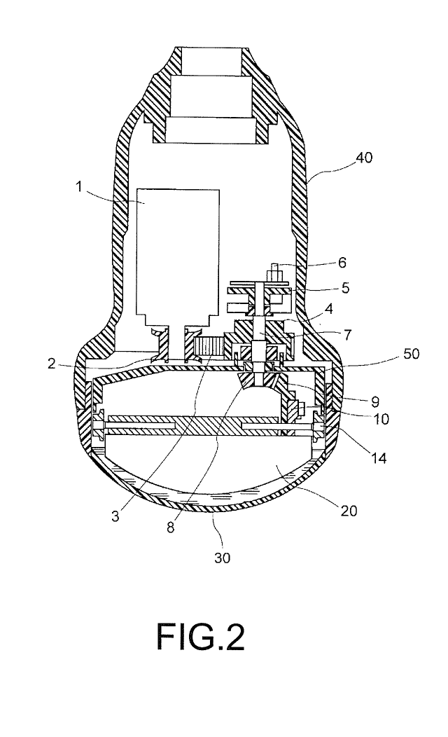

As shown in FIGS. 1 and 2, in an ultrasonic probe for medical diagnosis of the present invention, a housing is formed by a cap 30 of plastic material and a base 50 inserted in the cap 30, and an ultrasonic transmission and reception unit (piezoelectric element group) 20 with an acoustic lens is rotatably provided on a pair of rotary shafts 14 which are provided at a table 10 of a base 50 opposing to the other in a longitudinal direction of the ultrasonic probe. And liquid functioning as acoustic medium L, e.g., oil, is placed in the housing and sealed by covering the housing with a grip case 40 which is an exterior member made of the plastic material.

Then, a drive motor 1 provided in the grip case 40 is driven by supplying power from a power supply cable 60 so that the ultrasonic transmission and reception unit (piezoelectric element group) 20 oscillates, and ultrasonic waves transmitted and received from the ultrasonic transmission and reception surface of the ultrasonic transmission and reception unit 20 is mechanically scanned in a short-axis of the ultrasonic transmission and reception unit (piezoelectric element group) 20, thereby taking in three dimensional data for ultrasonic diagnosis of a subject.

Here, an oscillating mechanism of the ultrasonic transmission and reception unit (piezoelectric element group) of the ultrasonic probe of the present invention will be described with reference to FIGS. 2, 3 and 4.

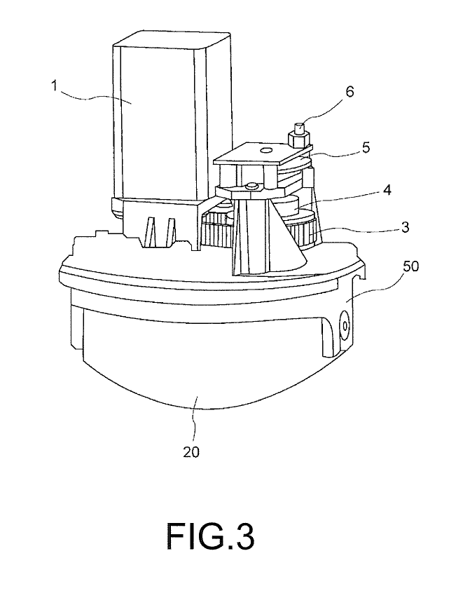

As shown in FIG. 2 and FIG. 3, it is constituted that the drive motor 1 is provided vertically at the upper surface of the base 50 constituting a part of the housing of the ultrasonic probe of the present invention, and that driving power of a motor pulley 2 fitted in a drive shaft extending from the lower end of the drive motor 1 is transmitted to a drive shaft pulley 4 fitted in a drive shaft 7 vertically and rotatably provided on the upper surface of the base 50, through a timing belt 3.

Further, a small bevel gear 8 is fitted in the drive shaft 7 at the lower end which is an output side of the ultrasonic probe, and a large bevel gear 9 meshing with the small bevel gear 8 is fitted in one of the rotary shafts 14 provided at the base plate 10, so that the rotation of the small bevel gear 8 is transmitted to the large bevel gear 9 to reduce the rotation of the drive shaft 7 and to change the rotating direction, thereby oscillating the ultrasonic transmission and reception unit (piezoelectric element group) 20.

Here, a reflector 5 is fitted in the upper end of the drive shaft 7, and a reflection type photo sensor 6 provided and fixed above the reflector 5 detects reference position of the ultrasonic transmission and reception unit (piezoelectric element group) 20.

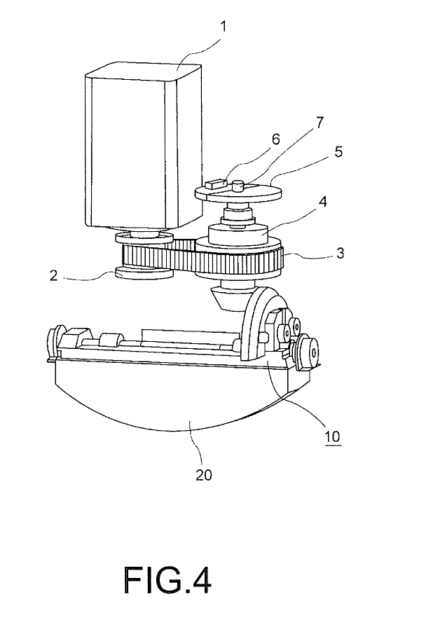

Further, the oscillating operation of the ultrasonic transmission and reception unit (piezoelectric element group) 20 as shown in FIG. 4 is controlled by the drive motor 1 itself, but may be controlled by a stepping motor which is controlled by open loop. Alternatively, it can be controlled by a DC motor or an AC motor, which is controlled by closed loop. In this case, for closed loop control, an encoder not shown here is provided.

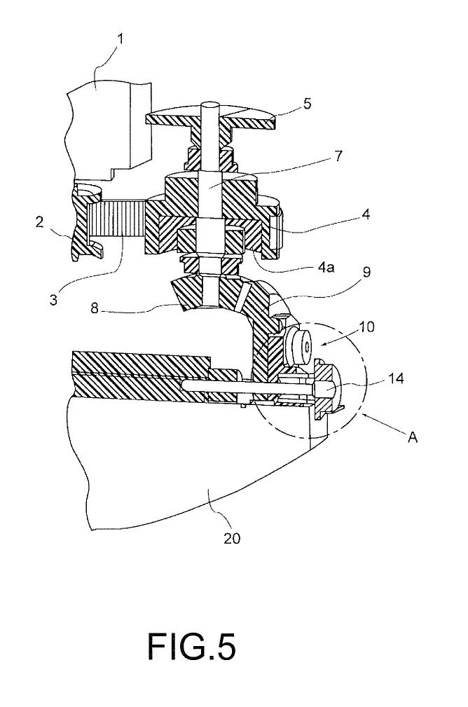

As shown in FIG. 5, a solid oil seal may be provided between an inner hollow part of the drive shaft pulley 4 and an outside surface of the drive shaft 7.

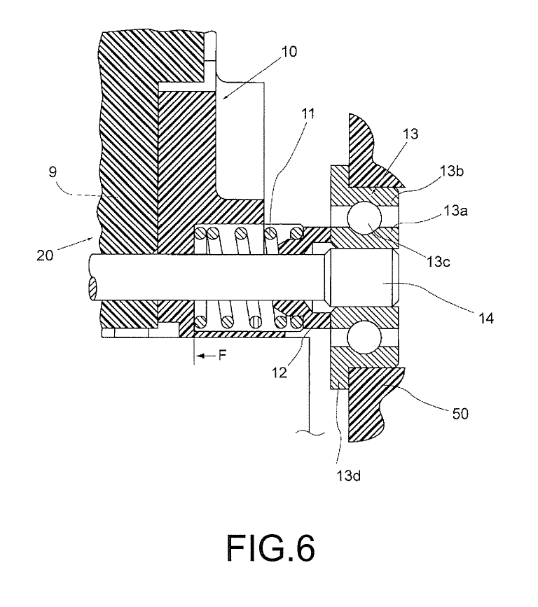

In particular, in the oscillating mechanism of the ultrasonic transmission and reception unit (piezoelectric element group) of the ultrasonic probe of the present invention, as shown in FIG. 6, the large bevel gear 9 oscillated by the rotation of the small bevel gear 8 is fixed at the rotary shaft 14, and the distal end portion of the rotary shaft 14 is rotatably supported to the base 50 by a ball bearing 13.

Further, a coiled compression spring 11 is arranged between the base plate 10 and a collar 12 which is slidably fitted in the rotary shaft 14 and pressed against the base plate 10 so as to generate pressing force to the base plate 10. Thus, since the compression spring 11 is regulated to move to a right direction in FIG. 6 by the base 50 through the collar 12 and the ball bearing 13, the compression spring 11 presses (F) the whole of the ultrasonic transmission and reception unit 20 to a left direction in FIG. 6 through the base plate 10.

Therefore, since the large bevel gear 9 is pressed toward the tooth surface of the small bevel gear 8 meshing with the large bevel gear 9, backlash does not occur between tooth surfaces of the bevel gears 8 and 9 even the ultrasonic transmission and reception unit 20 is in any oscillating position. In result, the work for adjusting backlash by hand is not needed.

Further, since the elastic force of the compression spring 11 acts between the base plate 10 and the base 50 through the ball bearing 13, it can reduce increase of frictional load during the oscillation of the ultrasonic transmission and reception unit 20.

In other words, the collar 12 is freely rotated and moved in an axial direction relative to the rotary shaft 14, and one end of the collar 12 is in contact with the compression spring 11 and the other end is in contact with an inner ring 13a of the ball bearing 13, and the inner ring 13a is freely rotated by a ball 13c relative to an outer ring 13b but the axial movement of the inner ring 13a is fixed, and further, a flange 13d of the outer ring 13c is engaged and fixed with the base 50. Here, the rotary shaft 14 is fixed to the base 10 while being freely moved in the axial direction relative to the inner ring 13a.

Further, an outer diameter that the inner ring 13c of the ball bearing 13 is fitted in the distal end portion of the rotary shaft 14 is larger than an outer diameter that the collar 12 is slidably fitted in the rotary shaft 14. Furthermore, since the outer portion that the collar 12 is slidably fitted in the rotary shaft 14 extends in the axial direction of the rotary shaft 14 and fixed at the base plate 10 with a predetermined length, the rotary shaft 14 is supported by the base plate 10 and the base 50 without axial shift.

Therefore, during assembly operation of the ultrasonic probe, it can be prevented that the collar 12 is discretely sprung by elastic force of the compression spring 11, and thus, assemblability of the ultrasonic probe is improved.

### Second Embodiment

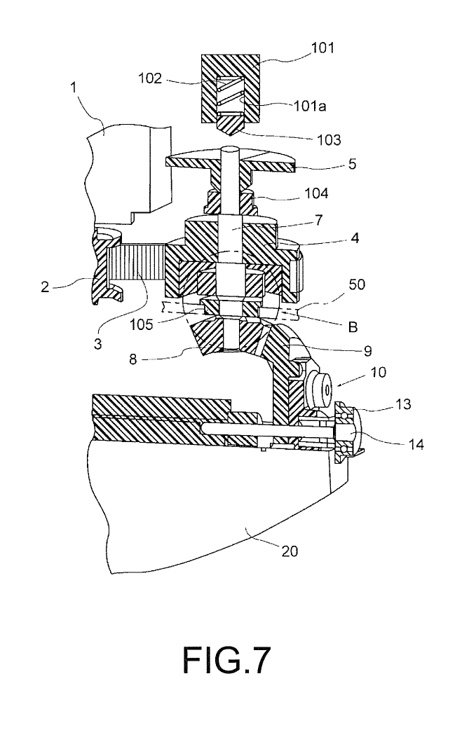

In the second embodiment of the ultrasonic probe of the present invention, as shown in FIG. 7, a holding frame 101, e.g., having a cylindrical shape is bridged above the upper end of the drive shaft 7 which rotates a small bevel gear 8, a compression spring 102 is held in a hole portion having a circular cross section formed in the holding frame 101, and a piece 103 is held in the hole portion 101a so as to freely move in the axial direction.

Here, a tip portion of the piece 103 is formed in a tapered shape or a spherical shape, so that the piece 103 presses the axial center of the upper end portion of the drive shaft 7 in point contact.

Because of this shape, even the pressing and elastic force of the compression spring 102 acts on the upper end of the drive shaft 7, the frictional force that prevents rotation of the drive shaft 7 does not occur.

In the second embodiment of the ultrasonic probe of the present invention, the drive shaft 7 which rotates the small bevel gear 8 is rotatably supported by a ball bearing 104 at the upper end portion and a ball bearing 105 at the lower end portion.

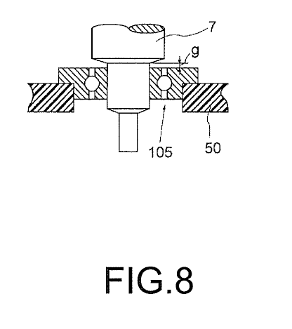

In particular, in the second embodiment of the ultrasonic probe of the present invention, as shown in FIG. 8 showing the enlarged cross sectional view pointed by an arrow B of FIG. 7, there is a gap g formed so that a stepped portion of the drive shaft 7 does not abut against the end surfaces of the inner ring and the outer ring of the ball bearing 105, and thus, the biasing and pressing force of the compression spring 102 acting on the drive shaft 7 is effectively transferred to the small bevel gear 8, and the tooth surface of the small bevel gear Bis constantly biased toward the tooth surface of the large bevel gear 9 meshing with the small bevel gear 8.

As a result, backlash between the tooth surfaces of the bevel gear 8 and 9 is eliminated.

### DESCRIPTION OF THE REFERENCE NUMERALS

1 drive motor
2 motor pulley
3 timing belt
4 drive shaft pulley
5 reflector
6 reflection type photo sensor
7 drive shaft
8 small bevel gear
9 large bevel gear
10 base plate
11 compression spring
12 collar
13 ball bearing
14 rotary shaft
20 ultrasonic transmission and reception section
30 cap
40 grip case
50 base
60 power supply cable

1. An ultrasonic probe, comprising:
an ultrasonic transmission and reception unit provided inside a housing, an acoustic transmission medium sealed in the housing, and a drive device for oscillating the ultrasonic transmission and reception unit;
wherein the drive device being a drive transmission mechanism for converting rotation of a drive motor to oscillation of the ultrasonic transmission and reception unit;
all or a part of the drive transmission mechanism comprising a gear mechanism;
in a meshing section of at least one pair of gears in the gear mechanism, one of the pair of gears being elastically biased and pressed to the other of the pair of gears;
the pair of gears being bevel gears meshing each other;
the one of the pair of bevel gears, together with other member that rotates integrally therewith being elastically biased and pressed to the other of the pair of bevel gears in a rotary shaft direction of the other member.

2. (canceled)

3. (canceled)

4. An ultrasonic probe according to claim 1, wherein
the other member that rotates integrally with the one of the pair of bevel gears is a drive shaft for transmitting rotational force to the one of the pair of bevel gears.

5. An ultrasonic probe according to claim 1, wherein
the ultrasonic transmission and reception unit is axially supported by a rotary shaft so as to freely oscillate; and the other member that rotates integrally with the one of the pair of bevel gears is the rotary shaft.

6. An ultrasonic probe according to claim 1, wherein
a member for biasing and pressing the one of the pair of bevel gears to the other of the pair of bevel gears is a compression spring provided around the other member that rotates integrally with the one of the pair of bevel gears.
